(19) United States
(12) Patent Application Publication
(10) Pub. No.: US 2007/0189973 A1
(43) Pub. Date: Aug. 16, 2007

(54) Contrast medium comprising liposomes containing hydrophobic chelate compound

(76) Inventor: Junji Nishigaki, Minami-Ashigara-shi (JP)

Correspondence Address: BIRCH STEWART KOLASCH & BIRCH, PO BOX 747, FALLS CHURCH, VA, 22040-0747, US

(21) Appl. No.: 10/579,912

(22) PCT Filed: Nov. 19, 2004
(86) PCT No.: PCT/JP04/17649

§ 371 Date: Feb. 15, 2007

(30) Foreign Application Priority Data
Nov. 21, 2003 (JP) 2003-392146

(51) Int. Cl. A61K 49/10

(52) U.S. Cl. 424/9.321

(57) ABSTRACT

A liposome containing a hydrophobic chelate compound as a membrane component, and an MRI contrast medium containing the liposome for use in imaging of, preferably, a vascular disease. An MRI contrast medium that accumulates in a lesion of a vascular disease caused by abnormal proliferation of vascular smooth muscle cells such as arteriosclerosis is provided.

### TECHNICAL FIELD

The present invention relates to an MRI contrast medium for arteriosclerotic lesions.

### BACKGROUND ART

In the modern society, especially in the societies of advanced countries, opportunities of ingesting high calorie and high fat diet are increasing. For this reason, mortalities due to ischemic diseases resulting from arteriosclerosis (heart diseases such as myocardial infarction and angina pectoris, cerebrovascular diseases such as cerebral infarction and cerebral hemorrhage) have been increasing. Therefore, it has been desired to diagnose such conditions at an early stage to employ an appropriate treatment. However, no satisfactory method is available for diagnosing progress of arteriosclerosis at an early stage before the onsets of the aforementioned diseases.

Methods for diagnosing arteriosclerosis are basically classified into non-invasive methods and invasive methods in which a catheter or the like is inserted into an artery. Among them, typical non-invasive methods include X-ray angiography and ultrasonography. However, by these methods, it is almost impossible to detect arteriosclerosis at an early stage, especially constriction of coronary artery, which causes myocardial infarction or angina pectoris, at an early stage before the onset of these diseases.

As the invasive methods, intravascular echo, vascular endoscope and the like have been used. It is recognized that an arteriosclerotic lesion with a thickness as thin as 0.1 mm can be measured by these methods. However, for employment of these methods, it is necessary to arterially insert an ultrasonic oscillator or an endoscope attached to an end of a catheter, which may result in serious physical stress and heaviness as well as a risk of a patient. Therefore, although these methods have been used therapeutically for patients after the attack of myocardial infarction and the like or as secondary prophylaxis, they cannot be used for a diagnostic purpose to know as to presence or absence or a degree of progress of arteriosclerosis in a patient before onset.

Among the non-invasive methods, the method most widely used for identification of a lesion of arterial vasoconstriction is X-ray angiography. This method comprises the step of administration of a water-soluble iodine contrast medium to visualize vascular flows, and detecting a lesion at which the flows are obstructed. However, these methods can only detect a lesion where constriction progresses 50% or more and fail to detect a lesion before the onset of attack of an ischemic disease.

Separately from the above methods, attempts have also been reported in which a hydrophobic iodine contrast medium or a hydrophilic contrast medium is formulated for selective accumulation in a target lesion (Japanese Unexamined Patent Publication (Kokai) No. 2003-55196, International Patent Publications WO95/19186, WO95/21631, WO89/00812, British Patent No. 867650, WO96/00089, WO94/19025, WO96/40615, WO095/2295, WO98/41239, WO98/23297, WO99/02193, WO97/06132, U.S. Pat. Nos. 4,192,859, 4,567,034, 4,925,649, Pharm. Res., 16 (3), 420 (1999), J. Pharm. Sci., 72 (8), 898 (1983), Invest. Radiol., 18 (3), 275 (1983)). For example, Japanese Unexamined Patent Publication No. 2003-55196 discloses that a hydrophobic iodine compound can be accumulated in arteriosclerotic lesions of experimental animals by encapsulation of the iodine compound in liposomes. However, these methods require administration of a large amount of contrast medium for the purpose of selective contrast of vascular diseases, and thus a problem raises in expression of toxicity.

Recently, an NMR imaging method (MRI) which detects various pathological lesions as images has been focused as one of non-invasive and non-destructive clinical diagnostic methods. For ordinary MRI measurements, uses of an MRI reagent (a contrast medium) are often required to enhance a contrast between a pathological lesion and a normal tissue.

Major imaging parameters of MRI that can be controlled by using a contrast medium include spin-lattice relaxation time (T1) and spin-spin relaxation time (T2). For example, a paramagnetic chelate using manganese (2+), gadolinium (3+), or iron (3+) as a base agent decreases a spin-lattice relaxation time (T1) and thereby increases a signal intensity. An MRI contrast medium comprising magnetic/superparamagnetic grains as a base agent decreases a spin-spin relaxation time (T2) and causes the decrease of signal intensity.

Massive administration of a paramagnetic chelate or paramagnetic compound using dysprosium as a base material also decreases an MR signal intensity. The details of the MRI contrast media (some of them are under development or commercially available) are described in, for example, the review by D. D. Stark and W. G. Bradley, Magnetic Resonance Imaging, Mosby 1992, Chapter 14.

MRI contrast media currently often reported include hydrophilic chelate compounds such as GdDTPA, GDDOTA, GdHPDO3A, and GdDTPA-BMA. These hydrophilic chelate compounds are extracellularly distributed and excreted from the kidney. Such compounds are useful for, for example, visualizing pathological lesions in the central nervous system. Examples of agents further specific to organs and tissues include MnDPDP, GdBOPA, GdEOB-DTPA, paramagnetic porphyrin, polymer compounds, grains, and liposomes thereof.

Furthermore, liposomes encapsulating various paramagnetic metal ions and chelates have been reported. For example, small monolayer liposomes (small unilamellar vesicles, SUV), large monolayer liposomes (large unilamellar vesicles, LUV) and multilayer liposomes (multilamellar vesicles, MLV) having various lipid compositions, surface charging degrees, and sizes have been proposed as MRI contrast media (for example, S. E. Seltzer, Radiology, 171, p.19, 1989; S. E. Seltzer et al., Invest. Radiol., 23, p.131, 1988; C. Tilcock et al., Radiology, 171, p.77, 1989; C. Tilcock et al., Biochim. Biophys. Acta, 10222, p.181, 1990; E. C. Unger et al., Invest. Radiol., 25, p.638, 1990; E. C. Unger et al., Invest. Radiol., 23, p.928, 1988; E. C. Unger et al., Radiology, 171, p.81, 1989; E. C. Unger et al., Magn. Reson. Imaging, 7, p.417, 1989; and J. Vion-Dury et al., J. Pharmacol. Exp. Ther., 250, p.1113, 1989). However, although liposome MRI contrast media are abundantly reported, no product of liposome contrast medium has been launched in the marketed so far. No liposome MRI contrast medium has successfully been reached to a stage of later clinical trial.

As a contrast medium for obtaining an MRI image of arteriosclerosis, in particular, an early stage lesion, manganese(III)-α, β, γ, δ, -tetrakis(4-sulfophenyl)porphine chelate (hereinafter may be abbreviated as “Mn-TSPP”) has been proposed (S. Kim, Medical Journal of Osaka University, 42, 1, 1990). Further, Japanese Patent Unexamined Publication (KOKAI) No. 7-316079/1995 discloses a liposome preparation comprising liposomes consisting of neutral phospholipids and charged phospholipids and encapsulating a water-soluble contrast medium in the liposomes. However, Mn-TSPP and the liposomes described in Japanese Patent Unexamined Publication No. 7-316079/1995 give a low accumulation ratio in a phase of cholesterol, which is the major cause of arteriosclerosis, and therefore they cannot provide practically satisfactory images.

### DISCLOSURE OF THE INVENTION

An object of the present invention is to provide means for selectively accumulating an MRI contrast medium in a lesion of a vascular disease caused by abnormal proliferation of vascular smooth muscle cells such as arteriosclerosis and restenosis after PTCA. Another object of the present invention is to image a biological environment of a vascular disease or the like by using the aforementioned means.

The inventors of the present invention conducted various studies to achieve the foregoing objects, and as a result, they found that liposomes containing a chelate compound having a hydrophobic substituent as one of membrane components accumulated in vascular smooth muscle cells and foam macrophages, which are main components of arteriosclerotic lesion. The present invention was achieved on the basis of the aforementioned finding.

The present invention thus provides a liposome containing a hydrophobic chelate compound as a membrane component.

As preferred embodiments of the present invention, provided are the aforementioned liposome, which contains as a membrane component a phospholipid selected from the group consisting of phosphatidylcholines and phosphatidylserines, preferably a combination of a phosphatidylcholine and phosphatidylserine at a molar ratio of from 3:1 to 1:2; and the aforementioned liposome, which contains as a membrane component a chelate compound having at least one substituent having 10 or more carbon atoms.

From another aspect, the present invention provides an MRI contrast medium, which comprises the aforementioned liposome. As preferred embodiments of the invention, provided are the aforementioned MRI contrast medium, which is used for imaging of a vascular disease; and the aforementioned MRI contrast medium, which is used for imaging of vascular smooth muscle cells which are abnormally proliferated under an influence of foam macrophages, for example, for imaging of an arteriosclerotic lesion or restenosis after PTCA.

### BEST MODE FOR CARRYING OUT THE INVENTION

The membrane components of the liposome of the present invention are not particularly limited. According to a preferred embodiment, phospholipids selected from the group consisting of phosphatidylcholines (PC) phosphatidylserines (PS) are preferably used in combination as the membrane components. Preferred examples of phosphatidylcholines include, but not limited thereto, egg PC, dimyristoyl-PC (D.MPC), dipalmitoyl-PC (DPPC), distearoyl-PC (DSPC), dioleyl-PC (DOPC) and the like. Examples of the phosphatidylserines include those having lipid moieties similar to those of the phospholipids mentioned as preferred examples of the phosphatidylcholines. The molar ratio of PC and PS (PC:PS) used for accumulation of the liposomes in arteriosclerotic lesions is preferably, for example, from 3:1 to 1:2, more preferably 1:1.

Other preferred embodiments of the liposome of the present invention includes the liposome containing a phosphatidylcholine and a phosphatidylserine and further containing a phosphoric acid dialkyl ester as membrane components. The two alkyl groups constituting the dialkyl ester of phosphoric acid are preferably the same groups. Each group may contain 6 or more carbon atoms, preferably 10 or more carbon atoms, more preferably 12 or more carbon atoms. An upper limit of the carbon number of the alkyl group is not particularly limited, and the number is generally 24 or less. Preferred examples of the phosphoric acid dialkyl ester include, but not limited thereto, dilauryl phosphate, dimyristyl phosphate, dicetyl phosphate and the like. In this embodiment, preferred amount of the phosphoric acid dialkyl ester is from 1 to 50 mass %, preferably from 1 to 30 mass %, further preferably from 1 to 20 mass %, based on the total mass of phosphatidylcholine and phosphatidylserine.

The components of the liposome of the present invention are not limited to the aforementioned components, and other components may be added. Examples of such components include cholesterol, cholesterol esters, sphingomyelin, monosial ganglioside GM1 derivatives described in FEBS Lett., 223, 42 (1987); Proc. Natl. Acad. Sci., USA, 85, 6949 (1988) etc., glucuronic acid derivatives described in Chem. Lett., 2145 (1989); Biochim. Biophys. Acta, 1148, 77 (1992) etc., polyethylene glycol derivatives described in Biochim. Biophys. Acta, 1029, 91 (1990); FEBS Lett., 268, 235 (1990) and the like. However, the components are not limited to these examples.

The liposome contained in the contrast medium of the present invention can be prepared by any methods known in the field of the art. Examples of the preparation method are described in the references as general review of liposomes, which are mentioned above, as well as in Ann. Rev. Biophys. Bioeng., 9, 467 (1980), “Liopsomes” (Ed. by M. J. Ostro, MARCELL DEKKER, INC.) and the like. Specific examples include, but not limited thereto, the ultrasonication method, ethanol injection method, French press method, ether injection method, cholic acid method, calcium fusion method, freeze and thawing method, reverse phase evaporation method and the like. Size of the liposome of the present invention may be any of those obtainable by the aforementioned methods. Generally, a size in average may be 400 nm or less, preferably 200 nm or less. Structure of the liposome is not particularly limited, and may be unilamellar or multilamellar structure. It is also possible to formulate one or more kinds of appropriate drugs or other contrast media in the liposome.

The structure of the hydrophobic chelate compound used in the present invention is not particularly limited. Typically, hydrophobic chelate compounds represented by the following general formula (1):
A-L-B (1)
can be used

In the general formula (1), “A” may be a physiologically acceptable paramagnetic metal salt or chelate, or may contain a free radical group, preferably a nitroxide type-free radical group. When the paramagnetic agent is a free metal ion, a manganese (2+) salt is preferred. The chelate is preferably based on manganese (2+), gadolinium (3+), dysprosium (3+), or iron (3+), and can contain any one of the chelating agents disclosed in International Publication WO 91/10645, for example, NTA, EDTA, HEDTA, DTPA, DTPA-BMA, BOPTA, TTHA, NOTA, DOTA, DO3A, HP-DO3A, EOB-DTPA, TETA, HAM, DPDP, porphyrins, and derivatives thereo. “A” is preferably GdDTPA, GdDOTA, GdHPDO3A, or GdDTPA-BMA, most preferably GdDTPA.

In the general formula (1), “B” represents a substituent having 10 or more carbon atoms. In order to have the chelate compound stably exist in a lipid bilayer, the substituent is preferably hydrophobic, and for example, is preferably an alkyl group having from 18 to 40 carbon atoms (numerical ranges defined with “from-to” expression in the specification includes values of lower and upper limits). Further, the substituent may contain one or more heteroatoms such as an oxygen atom, nitrogen atom, or sulfur atom. In general, a substituent having the total number of oxygen atom and nitrogen atom of 10 or less is preferred. The heteroatoms may constitute a backbone of the substituent and/or may be contained in a side chain.

The substituent more preferably has a structure similar to that of lipid components constituting biological membranes. Preferred examples of the substituent that satisfy such requirements include, for example, the cholesterol derivatives described in J. Med. Chem., 25 (6), 618 (1982); J. Med. Chem., 24 (1), 5(1981); Appl. Radial. Isot., 37 (8), 907 (1986); Steroids, 44 (1), 85 (1984); Steroids, 14 (5), 575 (1969) and the like. As the cholesterol derivatives, those described in the aforementioned references are preferred, and cholesterol is particularly preferred.

L represents a divalent bridging group containing at least one heteroatom (the “heteroatom” referred to in this specification means an any atom other than carbon atom such as nitrogen atom, oxygen atom and sulfur atom) in the main chain. This bridging group may be a saturated group, or may contain one or more unsaturated bonds. The number of the heteroatoms in the main chain is not particularly defined. The number may preferably be 5 or less, more preferably 3 or less, and most preferably 1. This bridging group may contain, as a partial structure, a functional group containing a carbon atom adjacent to a heteroatom. Examples of the functional group containing an unsaturated moiety or a heteroatom contained in the bridging group include, for example, an alkenyl group, an alkynyl group, an ester group (including carboxylic acid ester, carbonic acid ester, sulfonic acid ester, and sulfinic acid ester), an amido group (including carbonamido, urethane, sulfonamido, and sulfinamido), an ether group, a thioether group, a disulfide group, an amino group, an imido group and the like. The aforementioned functional groups may further have one or more substituents. When two or more of functional groups exist, they may be the same or different.

Preferred examples of the partial structure of the divalent bridging group represented by “L” include an alkenyl group, an ester group, an amido group, an ether group, a thioether group, a disulfide group, and an amino group, and more preferred are an alkenyl group, an ester group and an ether group. The heteroatom contained in the main chain is preferably oxygen atom or sulfur atom, and oxygen atom is most preferred. The carbon number of “L” is preferably from 7 to 30, more preferably from 10 to 25, most preferably from 10 to 20. “L” may have one or more substituents. When “L” has a substituent, a halogen atom or an alkyl group is preferred as the substituent. Further, it is also preferred that “L” does not have any substituent.

Preferred embodiments of “L” are specifically exemplified below. However, the bridging group in the compounds of the present invention is not limited to these examples.
—(CH₂)ₙ—O—, —(CH₂)ₘ—S—CH₂—, —(CH₂)ₘ—(C═O)O—,
—(CH₂)ₘ—(C═O)NH—, —(CH₂)ₘ—O(C═O)—, —(CH₂)ₘ—NH(C═O)—,
—(CH₂)ₛ—NH(C═O)—(CH₂)₂—O—, —CH₂—CH═CH—(CH₂)ₜ—O—,
—(CH₂)ₘ—CH(CH₃)—O—

In the formulas, “n” represents an integer of from 10 to 20, “m” represents an integer of from 9 to 19, “s” represents an integer of from 8 to 18, and “t” represents an integer of from 7 to 17.

The hydrophobic chelate compounds represented by the general formula (1) can be synthesized by a known method. Preferred examples of the hydrophobic chelate compounds represented by the general formula (1) are mentioned below. However, the hydrophobic chelate compounds used in the present invention are not limited to these examples.

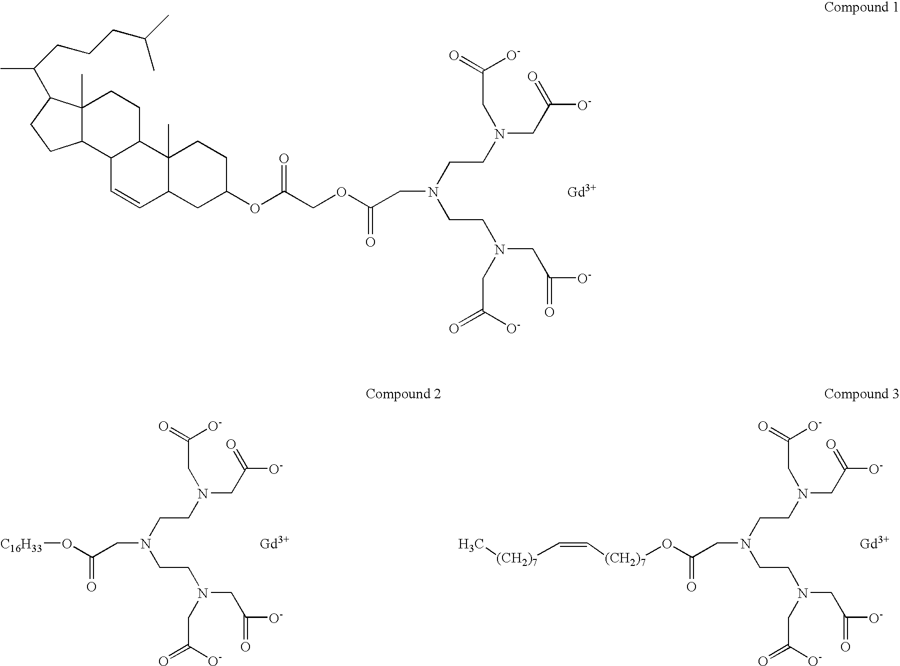

In the liposome of the present invention, the content of the hydrophobic chelate compound is about from 10 to 90 mass %, preferably from 10 to 80 mass %, more preferably from 20 to 80 mass %, based on the total mass of the membrane components of the liposome. One kind of the hydrophobic chelate compound may be used as a membrane component, or two or more kinds of the hydrophobic chelate compounds may be used in combination.

In the liposome containing a phosphatidylcholine, a phosphatidylserine, a phosphoric acid dialkyl ester and a hydrophobic chelate compound as membrane components according to a preferred embodiment of the present invention, preferred mass ratio of PC, PS, phosphoric acid dialkyl ester and hydrophobic chelate compound may be chosen from 5 to 40 mass %: from 5 to 40 mass %: from 1 to 10 mass %: from 15 to 80 mass %.

The MRI contrast medium of the present invention can be preferably administered parenterally, more preferably administered intravenously. For example, preparations in the form of an injection or a drip infusion can be provided as powdery compositions in a lyophilized form, and they can be used by being dissolved or resuspended just before use in water or an appropriate solvent (e.g., physiological saline, glucose infusion, buffering solution and the like). Concentration of the chelate compound in the liposome is, for example, in the range of from 1 mM to 0.5 M. A typical dose may be about 0.02 mM as the hydrophobic chelate compound per 1 kg of body weight. An optimum concentration and dose can be suitably chosen depending on the properties of the hydrophobic chelate compound, a route of administration, and clinical conditions such as a disease to be imaged and site of imaging.

Although it is not intended to be bound by any specific theory, it is known that, in vascular diseases such as arteriosclerosis or restenosis after PTCA, vascular smooth muscle cells constituting tunica media of blood vessel abnormally proliferate and migrate into endosporium at the same time to narrow blood flow passages. Although triggers that initiate the abnormal proliferation of normal vascular smooth muscle cells have not yet been clearly elucidated, it is known that migration of macrophages into endosporium and foaming are important factors. It is reported that vascular smooth muscle cells then cause phenotype conversion (from constricted to composite type).

When the liposomes of the present invention are used, the hydrophobic chelate compound can be selectively taken up into the vascular smooth muscle cells abnormally proliferated under influences of foam macrophages. As a result, MRI becomes possible with high contrast between vascular smooth muscle cells of a lesion and a non-pathological site. Therefore, the contrast medium of the present invention can be suitably used particularly for imaging of vascular diseases. For example, imaging of arteriosclerotic lesion or restenosis after PTCA can be performed.

Further, as described in J. Biol. Chem., 265, 5226 (1990), for example, it is known that liposomes containing phospholipids, particularly liposomes formed by using PC and PS, likely to accumulate on macrophages with the aid of scavenger receptors. Therefore, by using the liposomes of the present invention, the chelate compound of the present invention can be accumulated in a tissue or a lesion in which macrophages localize.

Examples of tissues in which localization of macrophages is observed, which can be suitably imaged by the method of the present invention, include blood vessel, liver, spleen, air vesicle, lymph node, lymph vessel, and renal epithelium. Further, it is known that macrophages accumulate in lesions in certain classes of diseases. Examples of such diseases include tumor, arteriosclerosis, inflammation, infection and the like. Therefore, lesions of such diseases can be identified by using the liposomes of the present invention. In particular, it is known that foam macrophages, which take up a large amount of denatured LDL with the aid of scavenger receptors, accumulate in atherosclerosis lesions at an early stage (Am. J. Pathol., 103, 181 (1981); Annu. Rev. Biochem., 52, 223 (1983)). Therefore, by performing MRI after accumulation of the liposomes of the present invention in the macrophages, it is possible to identify locations of atherosclerosis lesions at an early stage, which is hardly achievable by other means.

### EXAMPLE

The present invention will be explained more specifically with reference to the. examples. However, the scope of the present invention is not limited to the following example.

### Test Example 1

### Uptake Amount of Chelate Compound by Vascular Smooth Muscle Cells

Dipalmitoyl-PC (Funakoshi, No. 1201-41-0225) and dipalmitoyl-PS (Funakoshi, No. 1201-42-0237) at the ratio shown below were dissolved in chloroform together with a hydrophobic chelate compound (Compound 1 specifically shown above as a preferred compound) in an eggplant-shaped flask according to the method described in J. Med. Chem., 25 (12), 1500 (1982) to form a uniform solution. Then, the solvent was evaporated under reduced pressure to form a thin membrane on the bottom of the flask bottom. This thin membrane was dried in vacuum, then added with an appropriate amount of 0.9% physiological saline (Hikari Pharmaceutical, No. 512) and subjected to ultrasonication (No. 3542 probe type oscillator, Branson, 0.1 mW) for 5 minutes to obtain a uniform liposome dispersion. The diameters of the liposomes in the resulting dispersion were measured by using WBC analyzer (A-1042, Nihon Kohden). As a result, the diameters were found to be 40 to 65 nm. The liposome preparation mentioned below, which was produced by the above method, was added to a mixed culture system of vascular smooth muscle cells and macrophage described in International Publication WO 01/82977. The cells were cultured at 37° C. under 5% CO₂ for 24 hours, and the amount of the chelate compound taken up into the vascular smooth muscle cells was quantified. The results are shown below. The hydrophobic compound of the present invention was efficiently taken up by vascular smooth muscle cells, and it can be clearly understood that the compound has superior properties as a component lipid of liposomes for MRI contrast medium.

PC: 50 nmol+PS: 50 nmol+Compound 1: 75 nmol

Amount taken up by vascular smooth muscle cells: 20 nmol/mg protein

### INDUSTRIAL APPLICABILITY

The liposomes of the present invention can achieve accumulation of the hydrophobic chelate compounds represented by the general formula (1) in vascular smooth muscle cells abnormally proliferating under influence of foam macrophages, and are useful as an MRI contrast medium for selective imaging of a lesion of a vascular disease caused by abnormal proliferation of vascular smooth muscle cells.

1. A liposome containing a hydrophobic chelate compound as a membrane component.

2. The liposome according to claim 1, wherein the hydrophobic chelate compound has at least one substituent having 10 or more carbon atoms.

3. The liposome according to claim 2, which contains a phosphatidylcholine and a phosphatidylserine as membrane components.

4. The liposome according to claim 3, wherein molar ratio of the phosphatidylcholine and the phosphatidylserine is from 3:1 to 1:2.

5. An MRI contrast medium, which comprises a liposome according to claim 1.

6. The MRI contrast medium according to claim 5, which is used for imaging of a tissue or lesion where macrophages localize.

7. The MRI contrast medium according to claim 6, wherein the tissue where macrophages localize is selected from the group consisting of tissues of liver, spleen, air vesicle, lymph node, lymph vessel, and renal epithelium.

8. The MRI contrast medium according to claim 6, wherein the pathological site where macrophages localize is selected from the group consisting of lesions of tumor, inflammation, and infection.
